(19) United States
(12) Patent Application Publication
(10) Pub. No.: US 2010/0203637 A1
(43) Pub. Date: Aug. 12, 2010

(54) Fluorescent cell markers

(76) Inventors: Elena Korchagina, Moscow (RU); Nicolai Bovin, Moscow (RU); Stephen Henry, Auckland (NZ)

Correspondence Address: NIXON & VANDERHYE, PC, 901 NORTH GLEBE ROAD, 11TH FLOOR, ARLINGTON, VA, 22203, US

(21) Appl. No.: 12/310,803

(22) PCT Filed: Sep. 6, 2007
(86) PCT No.: PCT/NZ2007/000256

§ 371 Date: Mar. 4, 2010

(30) Foreign Application Priority Data
Sep. 6, 2006 (NZ) 549742
Sep. 7, 2006 (NZ) 549740

(51) Int. Cl. C12N 5/02; C12N 5/00; C07F 5/02; C07D 311/82; C07F 9/02; C07C 229/00

(52) U.S. Cl. 435/375; 435/325; 548/405; 549/388; 554/84; 554/112

(57) ABSTRACT

The preparation and use of fluorescent cell markers of the structure F—S₁-S₂-L is described where F is a fluorophore, S₁-S₂ is a spacer linking F to L, and L is a diacyl lipid.

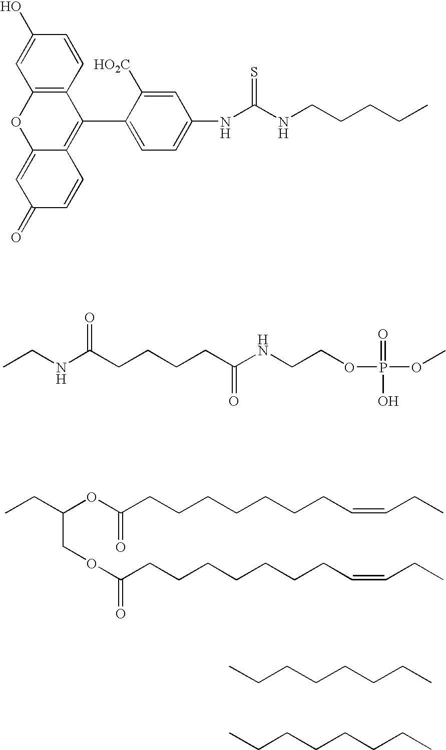

### TECHNICAL FIELD

The invention relates to fluorescent cell markers. In particular, the invention relates to fluorescent cell markers comprising the fluorophore of fluorescein, BODIPY, or one of their derivatives.

### BACKGROUND ART

The compounds fluorescein, BODIPY, and their derivatives comprise fluorophores.

Fluorescein is water soluble. Using fluorescein as a cell marker requires it to be conjugated to a reactive group such as isothiocyanate. The isothiocyanate group of fluorescein isothiocyanate (FITC) is reactive with the amine group of proteins.

FITC is used to label cells by conjugation with surface expressed proteins. The labeled cells may then be sorted by fluorescent-activated cell sorting (FACS).

The fluorophore of BODIPY has advantageous spectral characteristics over the fluorophore of fluorescein. Derivatives of BODIPY are also used in the labelling of cells by conjugation with surface expressed proteins.

Marking of cells by conjugation of a fluorophore with surface expressed proteins may affect cell function. Furthermore, mobility of the fluorophore within the two dimensions of the cell membrane is necessarily dependent on the mobility of the conjugated protein.

Alternative methods of marking cells that may avoid affecting cell function and provide for independent mobility of the fluorophore within the two dimensions of the cell membrane are therefore desired.

It is an object of this invention to provide an alternative method of marking cells or at least to provide a useful choice.

### DISCLOSURE OF INVENTION

In a first aspect the invention provides a fluorescent cell marker of the structure:

F—S₁-S₂-L

including the substructure:

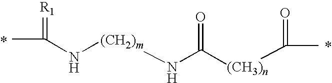

where
F is a fluorophore;
S₁-S₂ is a spacer linking F to L;
L is a lipid selected from the group consisting of diacyl- and dialkyl-glycerolipids, including glycerophospholipids;
m and n are independently 3 to 6;
R₁ is O or S; and
is other than H.

The spacer (S₁-S₂) is selected to provide a water soluble cell marker.

Preferably, F is selected from the group consisting of: fluorophores of fluorescein, Oregon Green, Pennsylvania Green, Tokyo Green, eosin, BODIPY, BODIPY TR, Alexa Fluor 350, Alexa Fluor 405, Alexa Fluor 488, Alexa Fluor 568, Alexa Fluor 594, Texas Red, Lucifer Yellow, tetramethylrhodamine and their derivatives. Most preferably, F is selected from the group consisting of: fluorophores of fluorescein, BODIPY and their derivatives.

Preferably, the sum of m and n is 6 to 9 and * is C or N.

Preferably, where F is the fluorophore of fluorescein or one of its derivatives, S₁ is a C₃₋₅-diaminoalkyl derivative selected from the group consisting of: 1,3-diaminopropyl, 1,4-diaminobutyl, or 1,5-aminopentyl derivatives. More preferably, where F is the fluorophore of fluorescein or one of its derivatives, S₁ is a C₃₋₅-aminoalkylthioureidyl. Most preferably, where F is the fluorophore of fluorescein or one of its derivatives, S₁ is 5-((5-aminopentyl) thioureidyl.

Preferably, where F is the fluorophore of fluorescein or one of its derivatives, S₂ is selected from the group including: —CO(CH₂)₃CO—, —CO(CH₂)₄CO— (adipate), —CO(CH₂)₅CO— and —CO(CH₂)₅NHCO(CH₂)₅CO—. More preferably, where F is the fluorophore of fluorescein or one of its derivatives, S₂ is —CO(CH₂)₄CO— (adipate).

Preferably, where F is the fluorophore of fluorescein or one of its derivatives, the structure includes the substructure:

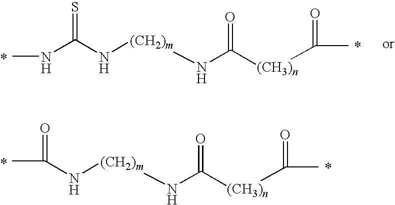

where m and n are independently 3 to 5 and * is other than H.

Preferably, where F is the fluorophore of BODIPY or one of its derivatives, S₁ is a C₃₋₅-alkionyldiamine. More preferably, where F is the fluorophore of BODIPY or one of its derivatives, S₁ is propionyl ethyldiamine.

Preferably, where F is the fluorophore of BODIPY or one of its derivatives, S₂ is selected from the group consisting of: —CO(CH₂)₃CO—, —CO(CH₂)₄CO— (adipate) and —CO(CH₂)₅CO—. More preferably, where F is the fluorophore of BODIPY or one of its derivatives, S₂ is —CO(CH₂)₄CO— (adipate).

Preferably, where F is the fluorophore of BODIPY or one of its derivatives the structure includes the substructure:

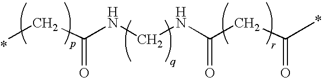

where p, q and r are independently 3 to 5 and * is other than H. More preferably, the sum of p, q and r is 8. Most preferably, p is 2, q is 2 and r is 4.

Preferably L is a lipid selected from the group consisting of diacyl- and dialkyl-glycerolipids, including glycerophospholipids. More preferably L is selected from the group consisting of: diacylglycerolipids, phosphatidate, phosphatidyl choline, phosphatidyl ethanolamine, phosphatidyl serine, phosphatidyl inositol, phosphatidyl glycerol, and diphosphatidyl glycerol derived from one or more of trans-3-hexadecenoic acid, cis-5-hexadecenoic acid, cis-7-hexadecenoic acid, cis-9-hexadecenoic acid, cis-6-octadecenoic acid, cis-9-octadecenoic acid, trans-9-octadecenoic acid, trans-11-octadecenoic acid, cis-11-octadecenoic acid, cis-11-eicosenoic acid or cis-13-docsenoic acid. More preferably the lipid is derived from one or more cis-desaturated fatty acids. Most preferably L is selected from the group consisting of: 1,2-O-dioleoyl-sn-glycero-3-phosphatidylethanolamine (DOPE), 1,2-O-distearyl-sn-glycero-3-phosphatidylethanolamine (DSPE) and rac-1,2-dioleoylglycerol (DOG).

In a first embodiment of the first aspect the invention provides a cell marker with the structure:

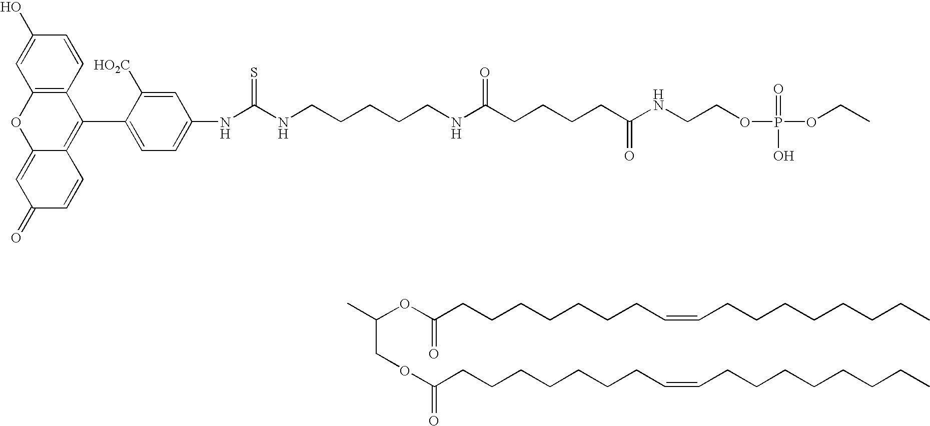

and designated KODE-fluorescein (I).

In a second embodiment of the first aspect the invention provides a cell marker with the structure:

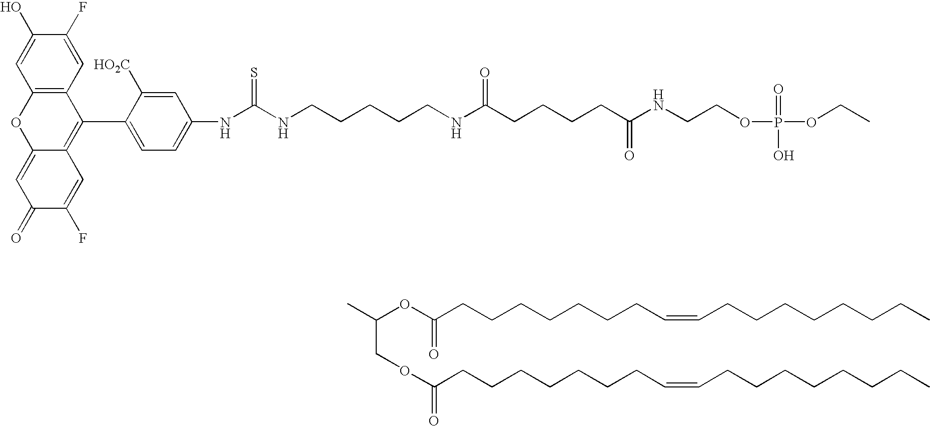

and designated KODE-Oregon Green (II).

In a third embodiment of the first aspect the invention provides a cell marker with the structure:

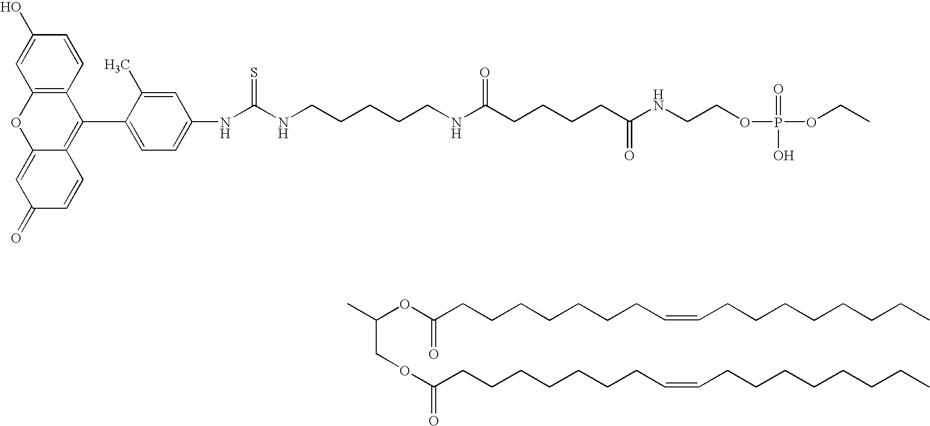

and designated KODE-Tokyo Green (III).

In a fourth embodiment of the first aspect the invention provides a cell marker with the structure:

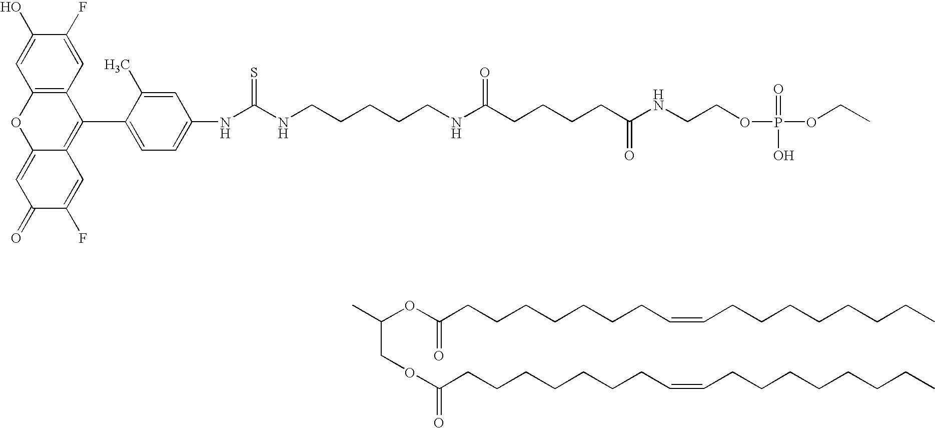

and designated KODE-Pennsylvania Green (IV).

In a fifth embodiment of the first aspect the invention provides a cell marker with the structure:

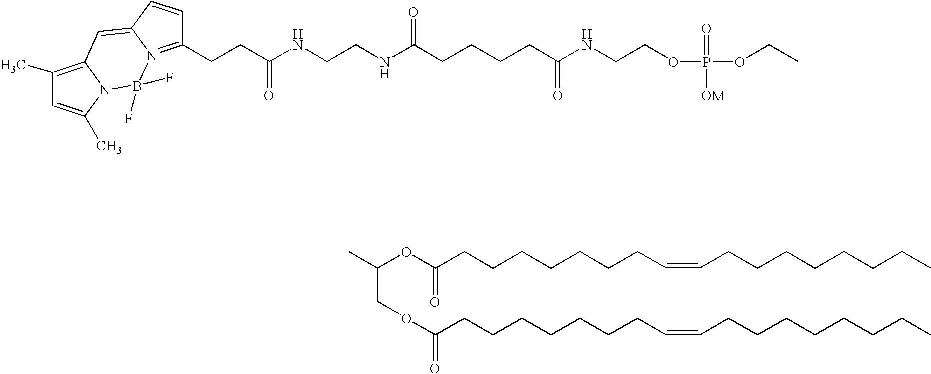

and designated KODE-BODIPY (V).

M is typically H, but may be replaced by another monovalent cation such as Na⁺, K⁺ or NH₄⁺.

In a second aspect the invention provides a method of marking cells including the step of:
Contacting a suspension of cells with a cell marker of the first aspect of the invention.

In a third aspect the invention provides a cell incorporating a cell marker of the first aspect of the invention.

In a fourth aspect the invention provides a cell produced by the method of the second aspect of the invention.

In the context of the description and claims:

“BODIPY” means the compound assigned the Chemical Abstracts Service (CAS) Registry number 138026-71-8 and the CA index name: Boron, difluoro[2-[(2H-pyrrol-2-ylidene-κN)methyl]-1H-pyrrolato-κN]-, (T-4)- (9CI).
“Fluorescein” means the chemical structure assigned the Chemical Abstracts Service (CAS) Registry number 518-47-8 and the CA index name: Spiro[isobenzofuran-1(3H), 9′-[9H]xanthen]-3-one, 3′,6′-dihydroxy-, sodium salt (1:2).
“Fluorophore” means the substructure or portion of a fluorescent molecule to which the fluorescent properties of the molecule are attributed.
“Or one of its derivatives” means a chemical modification of the chemical structure to provide a fluorophore with substantially equivalent physico-chemical properties, but modified spectral characteristics.
“Water soluble” means a stable, single phase system is formed when the cell marker is contacted with water or saline (such as PBS) in the absence of organic solvents or detergents, and the term “solution” has a corresponding meaning.

Exemplary embodiments of the invention will now be described with reference to the Figures of the accompanying drawings pages.

### BRIEF DESCRIPTION OF FIGURES

FIG. 1. Red blood cells following contact with cell marker (I) viewed with a fluorescence microscope at 470 nm under 250× magnification.

FIG. 2. Structure of cell marker designated KODE-fluorescein (I).

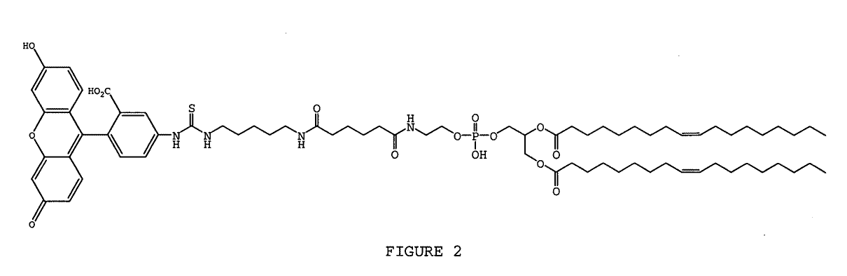

FIG. 3. Structure of cell marker designated KODE-Oregon Green (II).

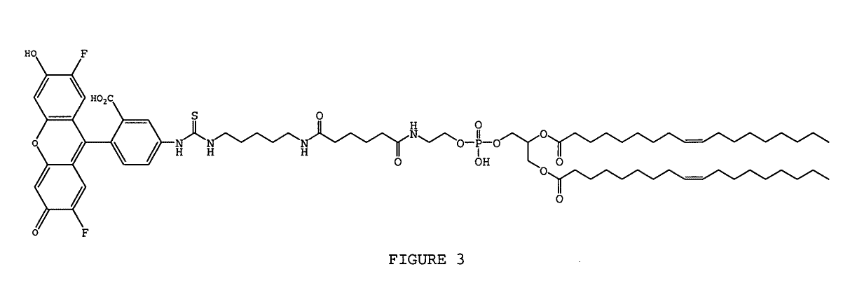

FIG. 4. Structure of cell marker designated KODE-Tokyo Green (III).

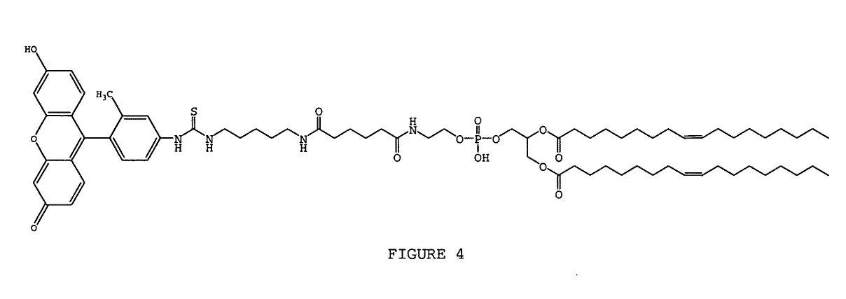

FIG. 5. Structure of cell marker designated KODE-Pennsylvania Green (IV).

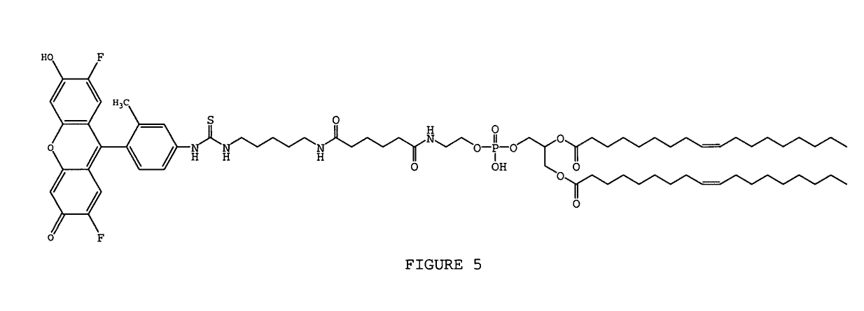

FIG. 6. Structure of cell marker designated KODE-BODIPY (V).

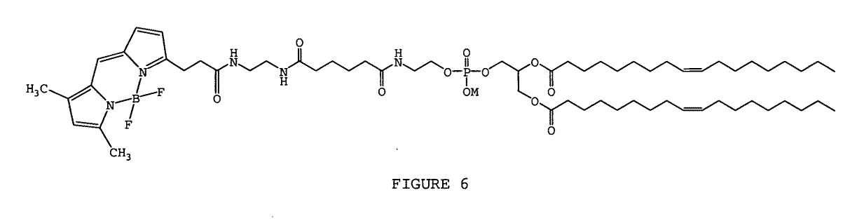

FIG. 7. ¹H-NMR spectrum of the cell marker designated KODE-BODIPY (V).

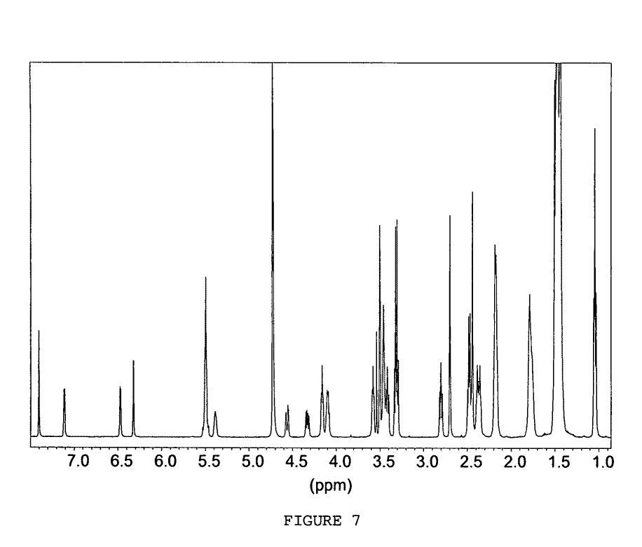

### DETAILED DESCRIPTION

The specification accompanying international application no. PCT/NZ2005/000052 (publication no. WO 2005/090368) describes water soluble synthetic molecules that are constructs of the structure F—S₁-S₂-L.

In these constructs F is a carbohydrate and the constructs spontaneously and stably incorporate into lipid bi-layers, including cell membranes.

The preferred constructs described in the specification accompanying the international application comprise the substructure:

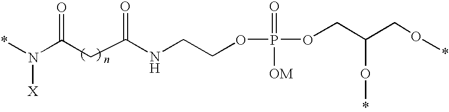

where n=3 to 5, X is H or C, and * is other than H.
M is typically H, but may be replaced by another monovalent cation such as Na⁺, K⁺ or NH₄⁺.
F is a fluorophore in the constructs of the present invention with different physicochemical properties to those of carbohydrate. The spacer (S₁-S₂) is selected to provide a construct that can be readily dispersed in aqueous vehicles such as saline.

Whilst not wishing to be bound by theory it is believed the cell markers of the present invention spontaneously incorporate into the lipid bi-layer of the cell membrane via their diacyl lipid tail. The fluorophore moiety is therefore expressed at the cell surface. The cell markers of the present invention can be used to mark cells without modification of the proteins expressed at the surface of the cell.

The likelihood of cell functions mediated by proteins expressed at the cell surface is reduced. Furthermore, the likelihood of the cell marker becoming uniformly distributed in the two dimensions of the lipid bilayer is increased. The mobility of the fluorophore is not dependent on the mobility of the cell surface expressed proteins to which the fluorophore might otherwise be conjugated.

Additional advantages are anticipated to accrue as the cell markers may allow studies on cell membrane dynamics independent of protein function and cycling. Cells labeled using the cell markers of the present invention may still be identified by conventional means and used in established biological methods such as fluorescence activated cell sorting (FACS) systems.

For the preparation of KODE-fluorescein (I), FITC is first conjugated with a diamine such as 1,5-diaminopentyl (cadaverine). The conjugated FITC is then reacted with an activated lipid (L-A) prepared as described in international application number PCT/NZ2005/000052.

A number of fluorescent compounds are available commercially as cadaverine derivatives. The cell markers where F is one of the fluorophores designated in Table 1 may be prepared.

TABLE 1
Fluorophores (represented as neutrally charged protonated species).

| Fluorophore | Designation |
|---|---|
| 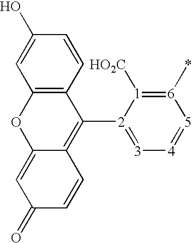 | Fluorescein (6-isomer) |
| 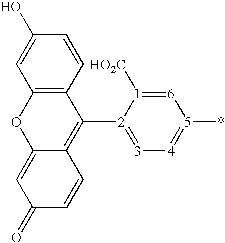 | Fluorescein (5-isomer) |
| 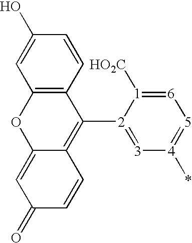 | Fluorescein (4-isomer) |
| 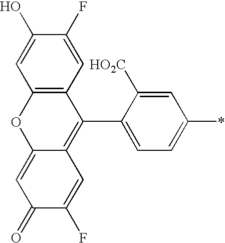 | Oregon Green (5-isomer) |
| 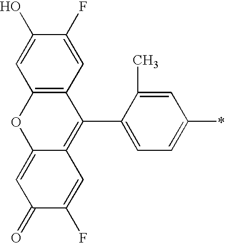 | Pennsylvania Green (5-isomer) |
| 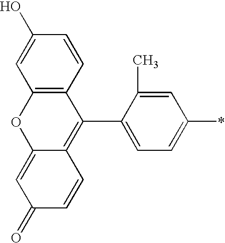 | Tokyo Green (5-isomer) |
| 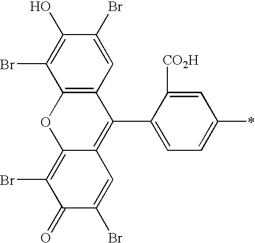 | Eosin (5-isomer) |
| 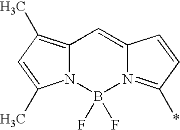 | BODIPY |
| 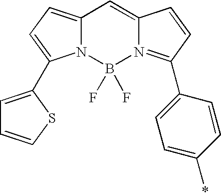 | BODIPY TR |
| 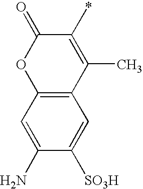 | Alexa Fluor 350 |
| 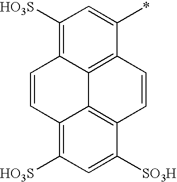 | Alexa Fluor 405 |
| 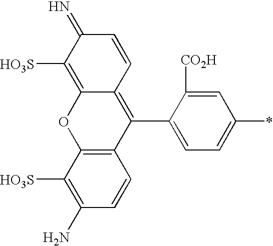 | Alexa Fluor 488 (5-isomer) |
| 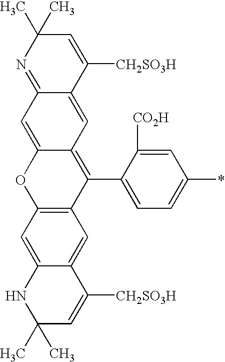 | Alexa Fluor 568 (5-isomer) |
| 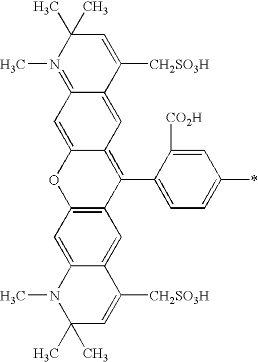 | Alexa Fluor 594 (5-isomer) |
| 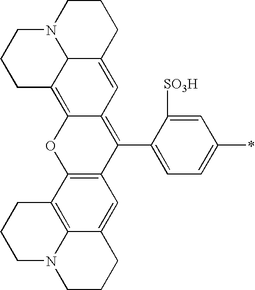 | Texas Red (5-isomer) |
| 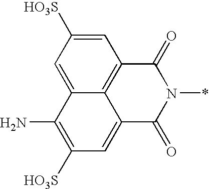 | Lucifer Yellow |
| 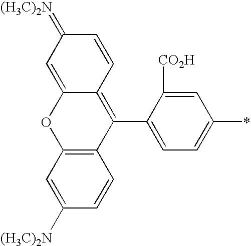 | Tetramethylrhodamine (5-isomer) |

For the preparation of KODE-BODIPY (V), BODIPY may alternatively be conjugated with an alkionyl diamine such propionyl ethylenediamine (BODIPY FL EDA). The conjugated BODIPY is then reacted with an activated lipid (L-A) prepared as described in the specification accompanying international application no. PCT/NZ2005/000052.

### Example 1

### Preparation of activated 1,2-O-distereoyl-sn-glycero-3-phosphatidylethanolamine (DSPE) and activated 1,2-O-dioleoyl-sn-glycero-3-phosphatidylethanolamine (DOPE)(L-A)

To a solution of bis(N-hydroxysuccinimidyl) adipate (A) (70 mg, 205 μmol) in dry N,N-dimethylformamide (1.5 ml) were added DOPE or DSPE (L) (40 μmol) in chloroform (1.5 ml) followed by triethylamine (7 μl). The mixture was kept for 2 h at room temperature, then neutralized with acetic acid and partially concentrated in vacuo.

Column chromatography (Sephadex LH-20, 1:1 chloroform-methanol, 0.2% acetic acid) of the residue yielded the activated lipid (L-A) (37 mg, 95%) as a colorless syrup; TLC (chloroform-methanol-water, 6:3:0.5): R_{f}=0.5 (DOPE-A), R_{f}=0.55 (DSPE-A).

¹H NMR (CDCl₃/CD₃OD, 2:1), δ:

DSPE-A—5.39 (m, 1H, —OCH₂—CHO—CH₂O—), 4.53 (dd, 1H, J=3.42, J=11.98, —CCOOHCH—CHO—CH₂O—), 4.33 (dd, 1H, J=6.87, J=11.98, —CCOOHCH—CHO—CH₂O—), 4.23 (m, 2H, PO—CH₂—CH₂—NH₂), 4.15 (m, 2H, —CH₂—OP), 3.61 (m, 2H, PO—CH₂—CH₂—NH₂), 3.00 (s, 4H, ONSuc), 2.81 (m, 2H, —CH₂—CO (Ad), 2.48 (m, 4H, 2×(—CH₂—CO), 2.42 (m, 2H, —CH₂—CO (Ad), 1.93 (m, 4H, COCH₂CH₂CH₂CH₂CO), 1.78 (m, 4H, 2×(COCH₂CH₂—), 1.43, 1.47 (2 bs, 40H, 20CH₂), 1.04 (m, 6H, 2CH₃).

DOPE-A—5.5 (m, 4H, 2×(—CH═CH—), 5.39 (m, 1H, —OCH₂—CHO—CH₂O—), 4.58 (dd, 1H, J=3.67, J=11.98, —CCOOHCH—CHO—CH2O—), 4.34 (dd, 1H, J=6.61, J=11.98, —CCOORCH—CHO—CH₂O—), 4.26 (m, 2H, PO—CH₂—CH₂—NH₂), 4.18 (m, 2H, —CH₂—OP), 3.62 (m, 2H, PO—CH₂—CH₂—NH₂), 3.00 (s, 4H, ONSuc), 2.8 (m, 2H, —CH₂—CO (Ad), 2.50 (m, 4H, 2×(—CH₂—CO), 2.42 (m, 2H, —CH₂—CO (Ad), 2.17 (m, 8H, 2×(—CH₂—CH═CH—CH₂—), 1.93 (m, 4H, COCH₂CH₂CH₂CH₂CO), 1.78 (m, 4H, 2×(COCH₂CH₂—), 1.43, 1.47 (2 bs, 40H, 20CH₂), 1.04 (m, 6H, 2CH₃).

### Condensation of DOPE-A with 5-((5-aminopentyl)thioureidyl) fluorescein (fluorescein cadaverine)

To a solution of activated DOPE (L-A) (5 mg, 5.2 μmol) in N,N-dimethylformamide (0.5 ml) 3 mg (4.6 μmol) of fluorescein cadaverine dihydrobromide salt and 5 μl of triethylamine were added. The mixture was kept for 2 h at room temperature, then 10 μl of 3% aq. NH₃ were added and the mixture was kept at room temperature for 1 h.

Column chromatography (Sephadex LH-20, 1:1 chloroform-methanol, followed by silica gel, ethyl acetate-isopropanol-water, 6:3:1) of the mixture yielded 4.2 mg (67%) KODE-fluorescein (I), R_{f} 0.5 (ethyl acetate-isopropanol-water, 6:3:1).

¹H NMR (CDCl₃/CD₃OD, 1:1), δ:

KODE-fluorescein (I)—8.38 (bs, 1H, aromatic proton of fluorescein), 8.15 (dd, 1H, J=1.7, J=8.3, aromatic proton of fluorescein) 7.30 (d, 1H, J=8.3, aromatic proton of fluorescein), 6.87 (m, 4H, aromatic protons of fluorescein), 6.72 (dd, 2H, J=2.4, J=8.8, aromatic protons of fluorescein), 5.50 (m, 4H, 2×(—CH═CH—), 5.38 (m, 1H, —OCH₂—CHO—CH₂O—), 4.58 (dd, 1H, J=6.6, J_{gem}=11.8, HHC—O—C(O)—), 4.34 (dd, 1H, J=3.2, J_{gem}=11.8, HHC—O—C(O)—), 4.14 (m, 2H, —OCH—CH₂—O—P—) (4.1 (m, 2H, —P—O—CH₂—CH₂—NH—) 3.80 (m, 2H, N—CH₂(CH₂)₃—CH₂NH—C═S) 3.39 and 3.58 (2m, 2×2H, N—CH₂—CH₂—O—P— and N—CH₂—(CH₂)₃—CH₂NH—C═S) 2.48 (m, 4H, 2×(—CH₂—CO), 2.39 (m, 4H, COCH₂CH₂CH₂CH₂CO), 2.19 (m, 8H, 2×(—CH₂—CH═CH—CH₂—), 1.84 (m, 2H, CH₂— fluorescein cadaverine), 1.8 (m, 10H, COCH₂CH₂CH₂CH₂CO, 2×(COCH₂CH₂—, and CH₂-fluorescein cadaverine), 1.62 (m, 2H, CH₂— fluorescein cadaverine) 1.42, 1.46 (2 bs, 40H, 20 CH₂), 1.05 (m, 6H, 2 CH₃).

Association of KODE-Fluorescein (I) with Cell Membranes

KODE-fluorescein (I) readily associates with the membrane of red blood cells. Insertion of the molecule is observed when dispersions of the molecule at concentrations greater than 0.1 mg/ml are contacted with suspensions of the red blood cells.

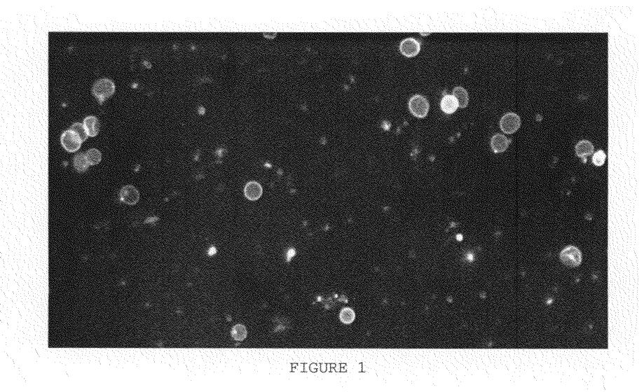

A medium to strongly fluorescing cell was considered to indicate a uniform distribution of the molecule across the cell membrane (FIG. 1). The incorporation and distribution appears to be stable for a period of at least 40 days when cells are stored in the dark.

### Example 2

Activated 1,2-O-dioleoyl-sn-glycero-3-phosphatidylethanolamine (DOPE) (L-A) was prepared as described in Example 1.

### Condensation of DOPE-A with 4,4-difluoro-5,7-dimethyl-4-bora-3a,4a-diaza-s-indacene-3-propionyl ethylenediamine, hydrochloride (BODIPY FL EDA)

To a solution of 15 mg (15.5 mmol) activated DOPE in CH₂Cl₂ (0.5 ml), 5 mg (13.5 mmol) of BODIPY FL EDA in N,N-dimethylformamide (0.3 ml) and 5 μl of triethylamine were added. The mixture was kept for 2 h at room temperature.

Column chromatography (Sephadex LH-20, 1:1 chloroform-methanol) of the mixture yielded 14.2 mg (75%) KODE-BODIPY (I), Et₃N-salt; MW 1289.6, R_{f} 0.3 (ethyl acetate-isopropanol-water, 6:3:1).

¹H NMR (CDCl₃/CD₃OD, 1:1): δ 7.40 (s, 1H, aromatic proton of BODIPY), 7.12 (d, 1H, J=3.8 aromatic proton of BODIPY), 6.47 (d, 1H, J=3.8 aromatic proton of BODIPY), 6.32 (s, 1H, aromatic protons of BODIPY), 5.50 (m, 4H, 2×(—CH═CH—), 5.38 (m, 1H, —OCH₂—CHO—CH₂O—), 4.58 (dd, 1H, J=3.2, J_{gem}=11.8, HHC—O—C(O)—), 4.33 (dd, 1H, J=6.6, J_{gem}=11.8, HHC—O—C(O)—), 4.16 (t, 2H, J=5.6, P—O—CH₂—CH₂—NH—), 4.1 (m, 2H, —OCH—CH₂—O—P—), 3.60 (t, 2H, P—O—CH₂—CH₂—NH—), 3.46, 3.42 and 2.8 (3m, 4H, 2H, 2H, —CH₂—CH₂—C(O)NH(CH₂)₂—NH of BODIPY), 2.70 (s, 3H, CH₃ of BODIPY), 2.48 (m, 4H, 2×(—CH₂—CO), 2.45 (s, 3H, CH₃ of BODIPY), 2.37 (m, 4H, COCH₂CH₂CH₂CH₂CO), 2.19 (m, 8H, 2×(—CH₂—CH═CH—CH₂—), 1.8 (m, 8H, COCH₂CH₂CH₂CH₂CO, 2×(COCH₂CH₂—)), 1.46, 1.43 (2 bs, 40H, 20 CH₂), 1.05 (m, 6H, 2 CH₃); 3.31 (q, 6H, J=7.4, 3×CH₂ of Et₃N), 1.50 (t, 9H, J=7.4, 3×CH₃ of Et₃N).

Although the invention has been described by way of exemplary embodiments it should be appreciated that variations and modifications may be made with out departing from the scope of the invention. Furthermore where known equivalents exist to specific features, such equivalents are incorporated as if specifically referred to in this specification.

1-30. (canceled)

31. A fluorescent cell marker of the structure:
F—S₁-S₂-L
including the substructure: 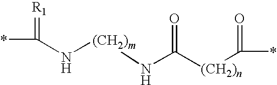
where:
F is a fluorophore;
S₁-S₂ is a spacer linking F to L;
L is a lipid selected from the group consisting of diacyl- and dialkyl-glycerolipids, including glycerophospholipids;
m and n are independently 3 to 6;
R₁ is O or S; and
* is other than H.

32. The fluorescent cell marker of claim 31 where F is selected from the group consisting of: fluorophores of fluorescein, Oregon Green, Pennsylvania Green, Tokyo Green, eosin, BODIPY, BODIPY TR, Alexa Fluor 350, Alexa Fluor 405, Alexa Fluor 488, Alexa Fluor 568, Alexa Fluor 594, Texas Red, Lucifer Yellow, tetramethylrhodamine and their derivatives.

33. The fluorescent cell marker of claim 31 where F is selected from the group consisting of: fluorophores of fluorescein, BODIPY and their derivatives.

34. The fluorescent cell marker of claim 31 where the sum of m and n is 6 to 9 and * is C or N.

35. The fluorescent cell marker of claim 31 where F is the fluorophore of fluorescein or one of its derivatives and S₁ is a C₃₋₅-diaminoalkyl derivative selected from the group consisting of: 1,3-diaminopropyl, 1,4-diaminobutyl, or 1,5-aminopentyl derivatives.

36. The fluorescent cell marker of claim 31 where F is the fluorophore of fluorescein or one of its derivatives and S₁ is a C₃₋₅-aminoalkylthioureidyl.

37. The fluorescent cell marker of claim 31 where F is the fluorophore of fluorescein or one of its derivatives and S₁ is 5-((5-aminopentyl) thioureidyl.

38. The fluorescent cell marker of claim 31 where F is the fluorophore of fluorescein or one of its derivatives and S₂ is selected from the group including: —CO(CH₂)₃CO—, —CO(CH₂)₄CO— (adipate), —CO(CH₂)₅CO— and —CO(CH₂)₅NHCO(CH₂)₅CO—.

39. The fluorescent cell marker of claim 31 where F is the fluorophore of fluorescein or one of its derivatives and S₂ is —CO(CH₂)₄CO— (adipate).

40. The fluorescent cell marker of claim 31 where F is the fluorophore of fluorescein or one of its derivatives and the structure includes the substructure: 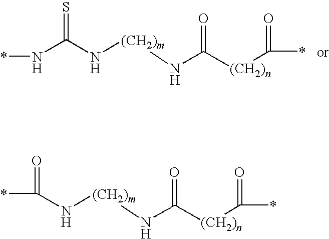
where m and n are independently 3 to 5 and * is other than H.

41. The fluorescent cell marker of claim 31 where F is the fluorophore of BODIPY or one of its derivatives and S₁ is a C₃₋₅-alkionyldiamine.

42. The fluorescent cell marker of claim 31 where F is the fluorophore of BODIPY or one of its derivatives and S₁ is propionyl ethyldiamine.

43. The fluorescent cell marker of claim 31 where F is the fluorophore of BODIPY or one of its derivatives and S₂ is selected from the group consisting of: —CO(CH₂)₃CO—, —CO(CH₂)₄CO— (adipate) and —CO(CH₂)₅CO—.

44. The fluorescent cell marker of claim 31 where F is the fluorophore of BODIPY or one of its derivatives and S₂ is —CO(CH₂)₄CO— (adipate).

45. The fluorescent cell marker of claim 31 where F is the fluorophore of BODIPY or one of its derivatives and the structure includes the substructure: 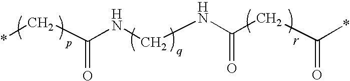
where p, q and r are independently 3 to 5 and * is other than H.

46. The fluorescent cell marker of claim 45 where the sum of p, q and r is 8.

47. The fluorescent cell marker of claim 46 where p is 2, q is 2 and r is 4.

48. The fluorescent cell marker of claim 31 where L is selected from the group consisting of: diacylglycerolipids, phosphatidate, phosphatidyl choline, phosphatidyl ethanolamine, phosphatidyl serine, phosphatidyl inositol, phosphatidyl glycerol, and diphosphatidyl glycerol derived from one or more of trans-3-hexadecenoic acid, cis-5-hexadecenoic acid, cis-7-hexadecenoic acid, cis-9-hexadecenoic acid, cis-6-octadecenoic acid, cis-9-octadecenoic acid, trans-9-octadecenoic acid, trans-11-octadecenoic acid, cis-11-octadecenoic acid, cis-11-eicosenoic acid or cis-13-docsenoic acid.

49. The fluorescent cell marker of claim 48 where the lipid is derived from one or more cis-desaturated fatty acids.

50. The fluorescent cell marker of claim 49 where L is selected from the group consisting of: 1,2-O-dioleoyl-sn-glycero-3-phosphatidylethanolamine (DOPE), 1,2-O-distearyl-sn-glycero-3-phosphatidylethanolamine (DSPE) and rac-1,2-dioleoylglycerol (DOG).

51. A fluorescent cell marker with the structure: 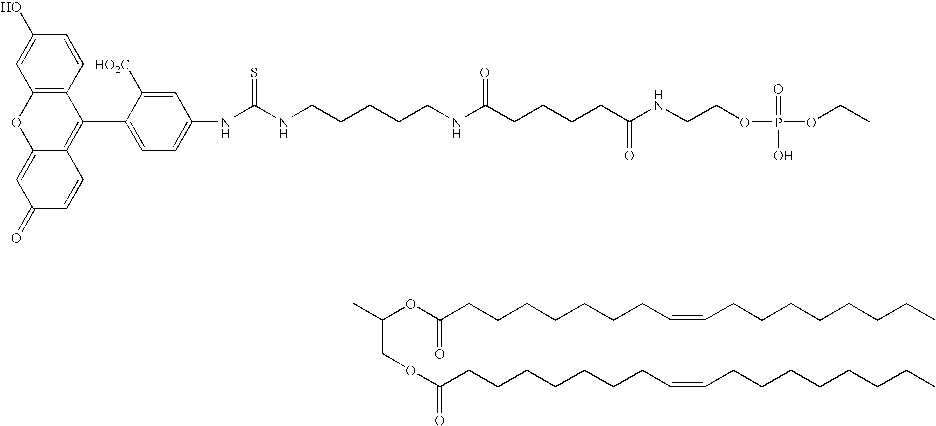
designated KODE-fluorescein (I) and where M is typically H, but may be replaced by another monovalent cation such as Na+, K+ or NH₄+.

52. A fluorescent cell marker with the structure: 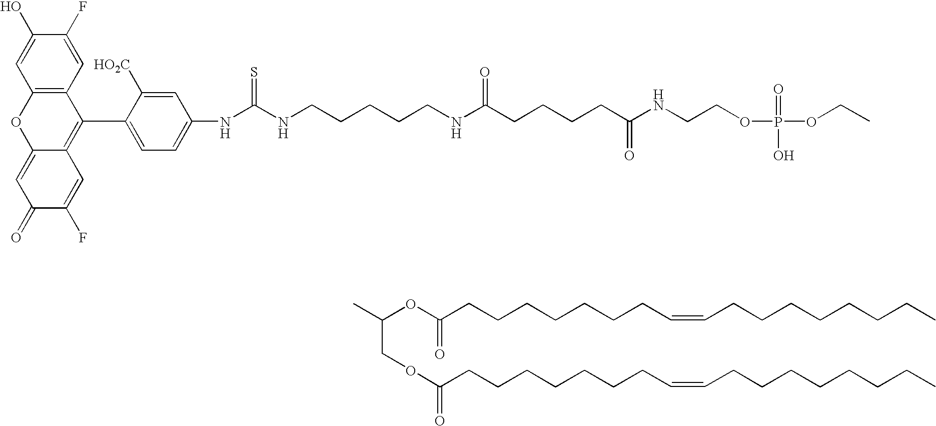
designated KODE-Oregon Green (II) and where M is typically H, but may be replaced by another monovalent cation such as Na+, K+ or NH₄+.

53. A fluorescent cell marker with the structure: 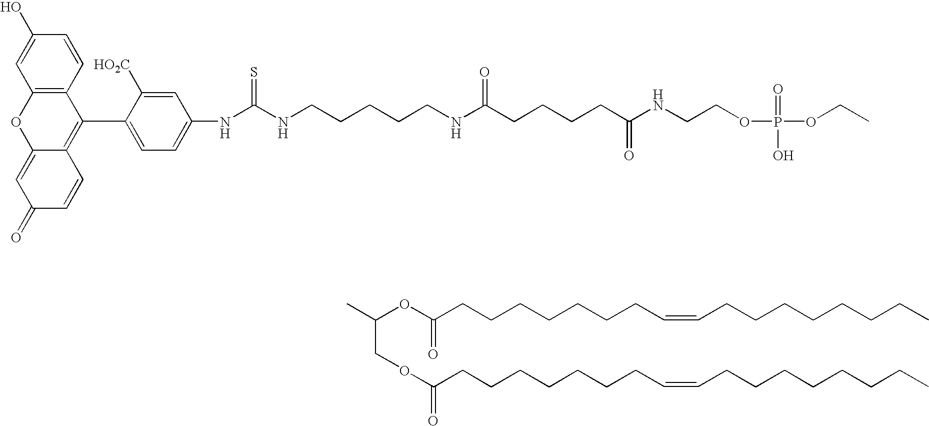
designated KODE-Tokyo Green (III) and where M is typically H, but may be replaced by another monovalent cation such as Na+, K+ or NH₄+.

54. A fluorescent cell marker with the structure: 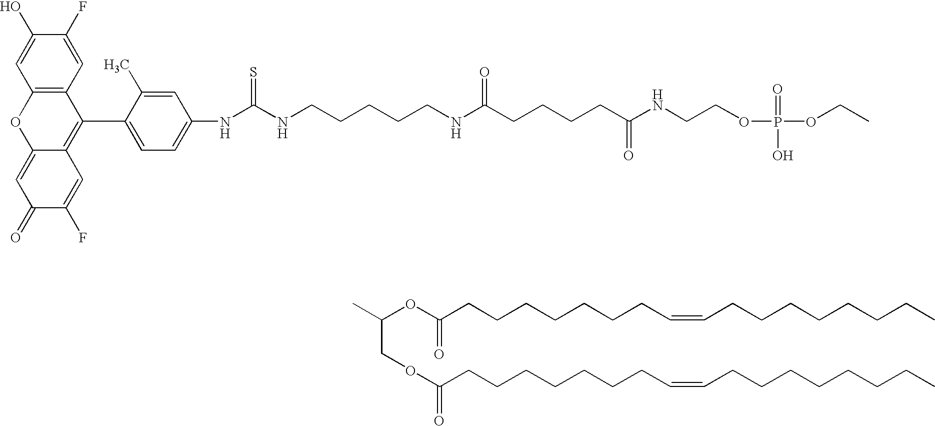
designated KODE-Pennsylvania Green (IV) and where M is typically H, but may be replaced by another monovalent cation such as Na+, K+ or NH₄+.

55. A fluorescent cell marker with the structure: 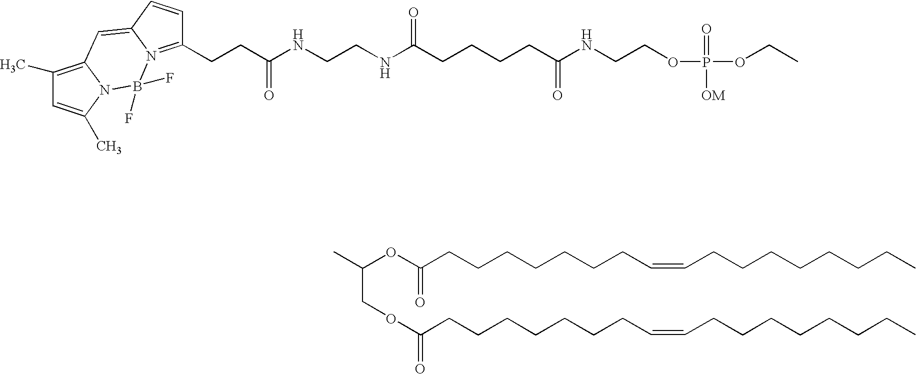
designated KODE-BODIPY (V) and where M is typically H, but may be replaced by another monovalent cation such as Na+, K+ or NH₄+.

56. A method of marking cells or multi-cellular structures including the step of:
Contacting a suspension of cells or multi-cellular structures with a cell marker of claim 31 for a time and at a temperature sufficient to allow incorporation of the marker into the membrane of the cell or multi-cellular structure.

57. A cell or multi-cellular structure incorporating a cell marker of claim 31.

58. A cell or multi-cellular structure produced by the method of claim 56.
